(19) United States
(12) Patent Application Publication
(10) Pub. No.: US 2020/0261033 A1
(43) Pub. Date: Aug. 20, 2020

(54) POSITIONING SYSTEM FOR RADIATION TREATMENT

(71) Applicant: Regain Biotech Corp., New Taipei (TW)

(72) Inventors: LIANG-HSIANG WU, New Taipei (TW); NGOT-SWAN CHONG, New Taipei (TW); CHIH-CHING CHANG, New Taipei (TW); YI-CHING LIU, New Taipei (TW); MING-HSUN HSU, New Taipei (TW)

(21) Appl. No.: 16/676,065

(22) Filed: Nov. 6, 2019

(30) Foreign Application Priority Data
Feb. 14, 2019 (TW) 108105005

(51) Int. Cl. A61B 6/04; A61B 5/055; A61B 6/00; A61B 5/00

(52) U.S. Cl. CPC A61B 6/04; A61B 5/68; A61B 6/54; A61B 5/0555

(57) ABSTRACT

A positioning system for positioning a body part of a patient for radiotherapy includes a positioning device and a radiation treatment system. The positioning device includes a photosensor disposed on an outer surface of the body part and configured to generate a sensing signal upon sensing a positioning beam. The processing unit is operable in a recording mode and a comparison mode. In the recording mode, the processing unit receives the sensing signal of the photosensor and generates reference positioning information according to the sensing signal. In the comparison mode, the processing unit receives the sensing signal of the photosensor and generates comparison positioning information according to the sensing signal. The processor compares the reference positioning information to the comparison positioning information and outputs a positioning result according to a result of comparison.

### FIELD

The subject matter herein generally relates to radiation treatment, and more particularly to a positioning system for positioning a body part of a patient for radiation treatment.

### BACKGROUND

Radiotherapy generally involves multiple stages. A first stage may be to locate a tumor in a body part of the patient. A positioning laser system of a tomography system is used to locate the tumor, and an operator manually marks a position of the tumor. A second stage may be to irradiate the tumor by a radiotherapy system. Therapeutic radiation is delivered at an isocenter of the radiation so as to minimize side effects of adjacent tissue. However, a position of the tumor is confirmed visually, which is prone to human error.

### BRIEF DESCRIPTION OF THE DRAWINGS

Implementations of the present disclosure will now be described, by way of embodiments, with reference to the attached figures.

FIG. 1 is a block diagram of an embodiment of a positioning system.

FIG. 2 is a diagram of a tomography system of the positioning system in FIG. 1.

FIG. 3 is a diagram of a radiotherapy system of the positioning system.

FIG. 4A is a diagram of reference positioning beams emitted to a plurality of photosensors.

FIG. 4B is a diagram of comparison positioning beams emitted to the plurality of photosensors.

FIG. 5 is a flowchart of an embodiment of a positioning method.

FIG. 6 is a flowchart of an embodiment of a positioning method.

### DETAILED DESCRIPTION

It will be appreciated that for simplicity and clarity of illustration, where appropriate, reference numerals have been repeated among the different figures to indicate corresponding or analogous elements. Additionally, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

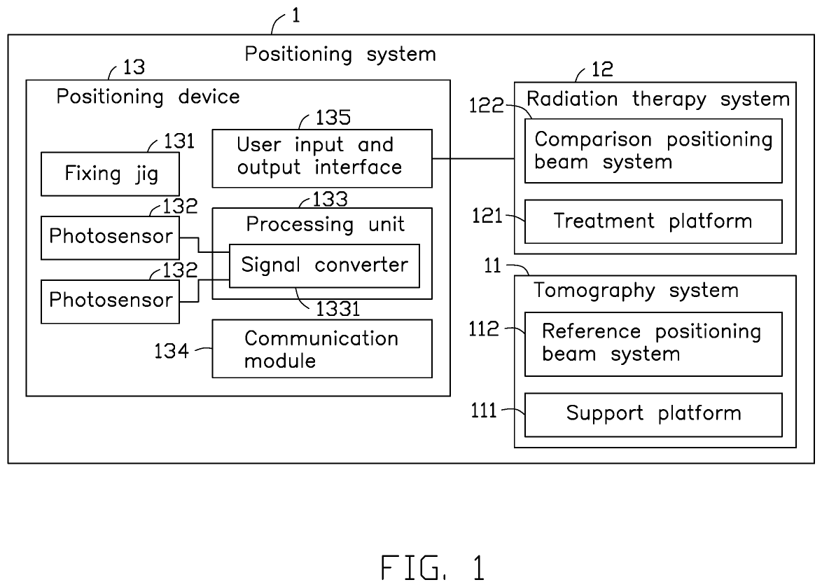

FIG. 1 shows a block diagram of an embodiment of a positioning system 1 for positioning a body part of a patient for radiotherapy. The positioning system 1 includes a tomography system 11, a radiotherapy system 12, and a positioning device 13.

The tomography system 11 can generate a tomographic image of a body part, such as an abdomen of a patient. In one embodiment, the tomographic image can be used to locate a tumor within the body part. The tomography system 11 may be a Computer Tomography (CT) system. In one embodiment, the tomography system 11 includes a support platform 111 and a reference positioning beam system 112. The support platform 111 can be used for a patient to lie down. The body part of the patient can be secured to the support platform 111 by a fixing jig that covers and conforms to a shape of the body part and can be secured on the support platform 111. The reference positioning beam system 112 is configured to generate one or more reference positioning beams. In one embodiment, when the patient is secured on the support platform 111, the reference positioning beams generated by the reference positioning beam system 112 can be used to position the body part of the patient.

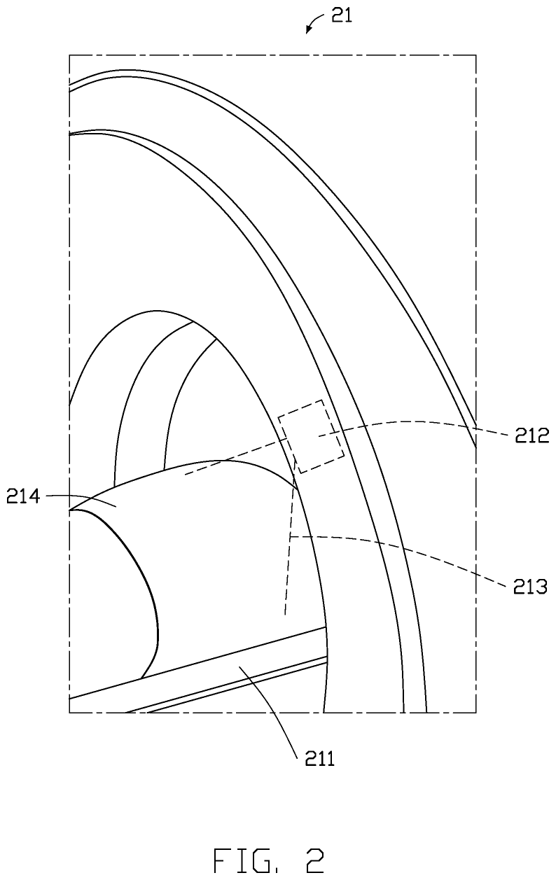

Referring to FIG. 2, a tomography system 21 includes a support platform 211 and a reference positioning beam system 212. The reference positioning beam system 212 is configured to emit reference positioning beams 213. The reference positioning beams 213 are emitted to the body part on the support platform 211. The tomography system 21 may have a configuration corresponding to the tomography system 11 in FIG. 1.

The radiotherapy system 12 is configured to radiate a tumor of the body with therapeutic radiation, which may be delivered to an isocenter of the radiation to minimize damage to adjacent tissue. The tumor receives a total dose of radiation to achieve the effect of treating cancer. In one embodiment, the radiotherapy system 12 includes a treatment platform 121 and a comparison positioning beam system 122. The treatment platform 121 can be used for patients to lie down. The comparison positioning beam system 122 is configured to generate comparison positioning beams. In some embodiments, the comparison positioning beams can be visible light or laser beams. In one embodiment, the patient can be secured to the treatment platform 121 by a fixing jig. In one embodiment, when the patient is secured to the treatment platform 121, the comparison positioning beams can be used to position the body part of the patient. The radiotherapy system 12 is further configured to move the treatment platform 121 based on the positioning results from the positioning device 13 to enable the patient to be moved to an appropriate treatment location. In one embodiment, the radiotherapy system 12 includes a processor (not shown) and a motor (not shown). The processor controls the motor to move the treatment platform 121 according to the positioning results.

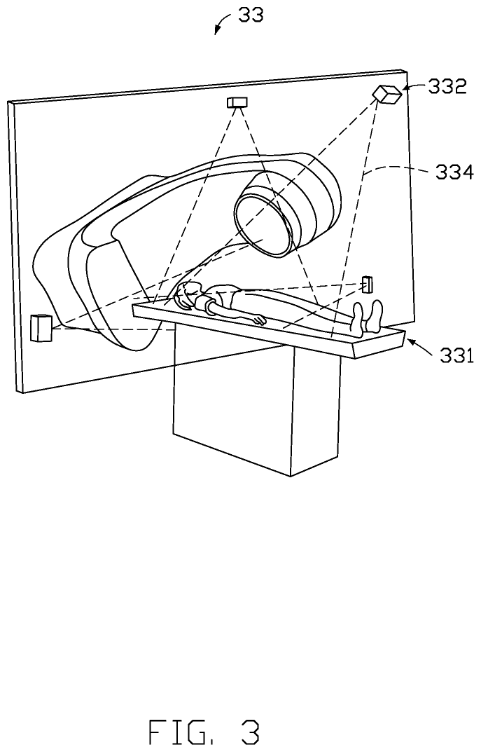

FIG. 3 shows an environment of a radiotherapy system 33 including a treatment platform 331 and a comparison positioning beam system 332. The comparison positioning beam system 332 can emit comparison positioning beams 334. The radiotherapy system 33 may have a configuration corresponding to the radiotherapy system 12 in FIG. 1.

In one embodiment, the positioning device 13 includes a fixing jig 131, a plurality of photosensors 132, a processing unit 133, a communication unit 134, and a user input and output interface 135. When the comparison positioning beam system 332 generates the comparison positioning beams, the processing unit 133 of the positioning device 13 determines whether the patient is in a suitable position according to the comparison positioning beams and selectively transmits a positioning result to the radiotherapy system 33 based on the determination.

The fixing jig 131 is used to fix a patient on a treatment platform. In one embodiment, the fixing jig 131 includes a radiotherapy thermoplastic mask. The fixing jig 131 includes an outer surface, which may be an outer surface of the radiotherapy thermoplastic mask.

The photosensors 132 may be flexible photosensors which are configured to be disposed on a surface of the body part of the patient, and each photosensor 132 is configured to generate a corresponding sensing signal upon sensing the reference positioning beams and the comparison positioning beams. The photosensors 132 can be disposed directly or indirectly on a surface of the patient. Directly disposed refers to a contact surface of the photosensor 132 being disposed on the surface of the body part, and indirectly disposed refers to the contact surface of the photosensor 132 being disposed on an outer surface of a conformable fixing jig (such as the fixing jig 131) that covers the patient. The photosensors 132 are disposed in a fixed arrangement and may be spaced in close proximity to each other or spaced further apart according to requirements.

The processing unit 133 includes a signal converter 1331. The signal converter 1331 is configured to convert the sensing signals generated by the photosensors 132 into digital signals. The processing unit 133 operates in a recording mode and a comparison mode.

It should be understood that the processing unit 133 may include other hardware modules, software modules, or firmware not described herein.

The reference positioning beam system 112 is configured to generate reference positioning beams when the processing unit 133 operates in the recording mode. When the processing unit 133 operates in the recording mode and receives the sensing signals from the photosensors 132, the processing unit 133 generates reference positioning information corresponding to the photosensors 132 that generated the sensing signals. The reference positioning information is used for reference for performing radiotherapy.

The comparison positioning beam system 122 is configured to generate comparison positioning beams when the processing unit 133 operates in the comparison mode. When the processing unit 133 operates in the comparison mode and receives the sensing signals from the photosensors 132, the processing unit 133 generates current positioning information corresponding to the photosensors 132 that generated the sensing signals and determines whether the reference positioning information of the corresponding photosensors 132 differs from the current positioning information of the corresponding photosensor 132. If the reference positioning information differs from the current positioning information of the corresponding photosensor 132, a message indicating positioning error is output to a display unit (not shown) for display. Thus, a positioning error and an adjustment direction can be confirmed, so that an operator can adjust the position accordingly.

When the processing unit 133 determines that the reference positioning information corresponding to the photosensors 132 is the same as the current positioning information, a message indicating positioning success is output to the display unit for display. Thus, a treatment site of the radiotherapy is ensured to be the same each time the radiotherapy is performed, thereby reducing human error and improving treatment accuracy.

In one embodiment, the positioning results can be output to a radiotherapy system (such as the radiotherapy system 12). In other embodiments, the positioning results may be output to the user input and output interface 135 for display.

The communication unit 134 is configured to communicatively couple the processing unit 133 and the radiotherapy system 12 and transmit the positioning results to the radiotherapy system 12. The communication unit 134 may be a wireless communication unit or a wired communication unit.

The user input and output interface 135 is coupled to the processing unit 133 and configured to transmit a mode switching instruction to the processing unit 133 via a human operation. The processing unit 133 is configured to operate in one of the recording mode and the comparison mode according to the mode switching instruction. In one embodiment, the user input and output interface 135 can include a touch display panel (not shown) to receive touch input for switching the processing unit 133 between the recording mode and the comparison mode.

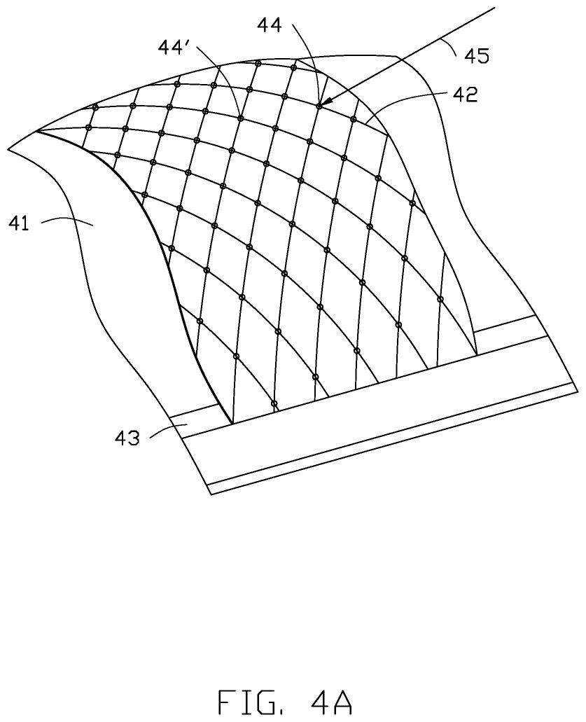

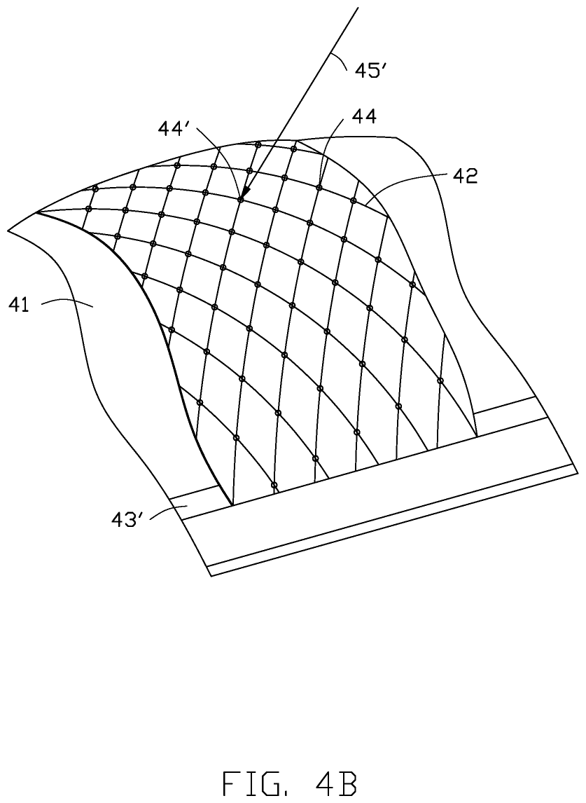

FIG. 4A shows a diagram of a patient undergoing a tomographic scan. A body part 41 is the patient's abdomen. A fixing jig 42 is a radiotherapy thermoplastic mask. A plurality of photosensors 44, 44′ are disposed on the fixing jig 42. The fixing jig 42 secures the body part 41 on a support platform 43 of a tomography system. At this time, a processing unit (such as the processing unit 133) coupled to the photosensors 44, 44′ can operate in the recording mode. As shown in FIG. 4A, the photosensor 44 senses a reference positioning beam 45 emitted from a reference positioning beam system (such as the reference positioning beam system 112) and generates a sensing signal in response to sensing the reference positioning beam 45. The processing unit generates reference positioning information corresponding to the photosensor 44 upon receiving the sensing information from the photosensor 44. In one embodiment, the reference positioning information may include, but is not limited to, identification information of the photosensor 44 and/or coordinate information of the photosensor 44. The patient is then ready to be moved to a radiotherapy room.

Referring to FIG. 4B, the patient is ready for radiotherapy, and the body part 41 is secured on a treatment platform 43′. At this time, the processing unit (such as the processing unit 133) operates in the comparison mode. As shown in FIG. 4B, the photosensor 44′ senses a comparison positioning beam 45′ emitted from a positioning beam system (such as the comparison positioning beam system 122) and generates a sensing signal in response to sensing the comparison positioning beam 45′. The processing unit generates current positioning information upon receiving the sensing information from the photosensor 44′. In one embodiment, the current positioning information may include, but is not limited to, identification information of the photosensor 44′ and/or coordinate information of the photosensor 44′.

As shown in FIGS. 4A and 4B, the comparison positioning beam 45′ does not emit on the photosensor 44. Thus, the reference positioning information differs from the current positioning information, so the radiotherapy system 12 moves the treatment platform 43′ so that the comparison positioning beam 45′ emits to the photosensor 44. In this way, a treatment site of the radiotherapy is ensured to be the same each time the radiotherapy is performed, thereby reducing human error and improving treatment accuracy.

It should be understood that the reference positioning beams and the comparison positioning beams are emitted from the same angle and position.

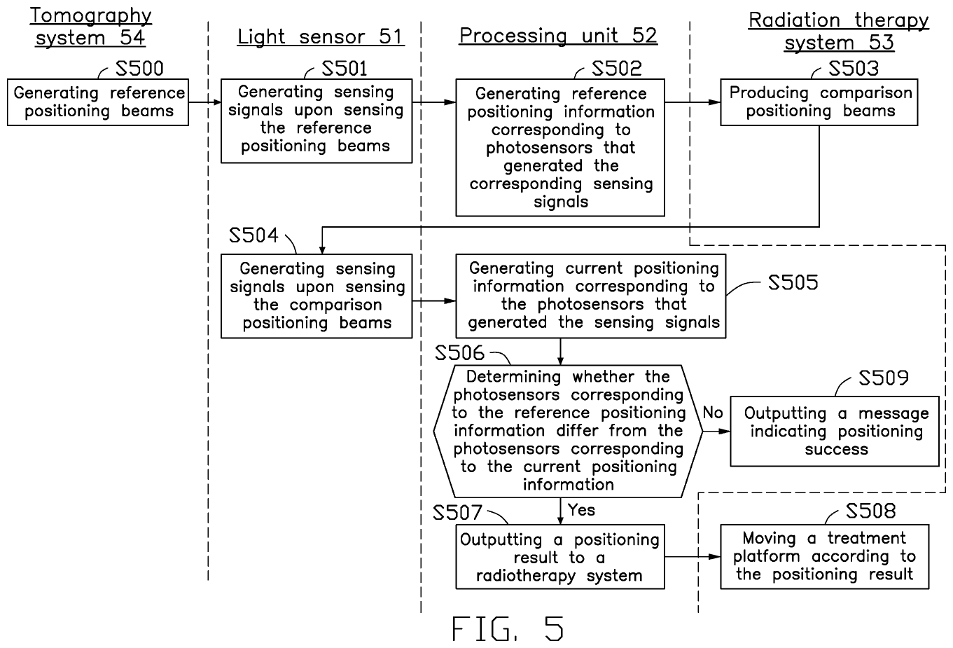

FIG. 5 shows a flowchart of an embodiment of a positioning method. The method is provided by way of embodiment, as there are a variety of ways to carry out the method. The method described below can be carried out using the configurations illustrated in FIGS. 1-4B, for example, and various elements of these figures are referenced in explaining the example method. Each block shown in FIG. 5 represents one or more processes, methods, or subroutines carried out in the example method. Furthermore, the illustrated order of blocks is by example only, and the order of the blocks can be changed. Additional blocks can be added or fewer blocks can be utilized, without departing from this disclosure.

At block S500, a tomography system 54 generates reference positioning beams. A plurality of photosensors 51 are disposed on a surface of a fixing jig securing a body part or a surface of skin of the body part. The body part is fixed on a support platform of the tomography system 54 by means of the fixing jig.

At block S501, the plurality of photosensors 51 generate sensing signals upon sensing the reference positioning beam.

At block S502, a processing unit 52, upon receiving the sensing signals from the photosensors, generates reference positioning information corresponding to the photosensors that generated the corresponding sensing signals. At this time, the processing unit 52 operates in a recording mode.

At block S503, a comparison positioning beam system of a radiotherapy system 53 produces comparison positioning beams. In one embodiment, the body part can be secured to a treatment platform of the radiotherapy system 53 by the same fixing jig.

At block S504, the photosensors 51 generate sensing signals upon sensing the comparison positioning beams.

At block S505, the processing unit 52 generates current positioning information corresponding to the photosensors 51 that generated the sensing signals upon receiving the sensing signals from the photosensors 51. At this time, the processing unit 52 operates in a comparison mode.

At block S506, the processing unit 51 determines whether the photosensors 51 corresponding to the reference positioning information differ from the photosensors 51 corresponding to the current positioning information. In some embodiments, the reference positioning information and the current positioning information include identification information of the photosensors 51. If the processing unit 52 determines that the photosensors 51 corresponding to the reference positioning information differ from the photosensors 51 corresponding to the current positioning information, block S507 is implemented. When the processing unit 52 determines that the photosensors 51 corresponding to the reference positioning information are the same as the photosensors 51 corresponding to the current positioning information, block S509 is implemented.

At block S507, the processing unit 52 outputs a positioning result to the radiotherapy system 53. The positioning result may include the reference positioning information and the current positioning information. In other embodiments, the processing unit 51 may also output the positioning result to a user input and output interface (such as the user input and output interface 135) for display to inform an operator, such as a radiotherapist.

At block S508, the radiotherapy system 53, upon receiving the positioning result from the processing unit 52, moves the treatment platform according to the positioning result.

At block S509, the processing unit 52 outputs a message indicating positioning success to a user input and output interface (such as the user input and output interface 135) for display to inform an operator, such as a radiotherapist.

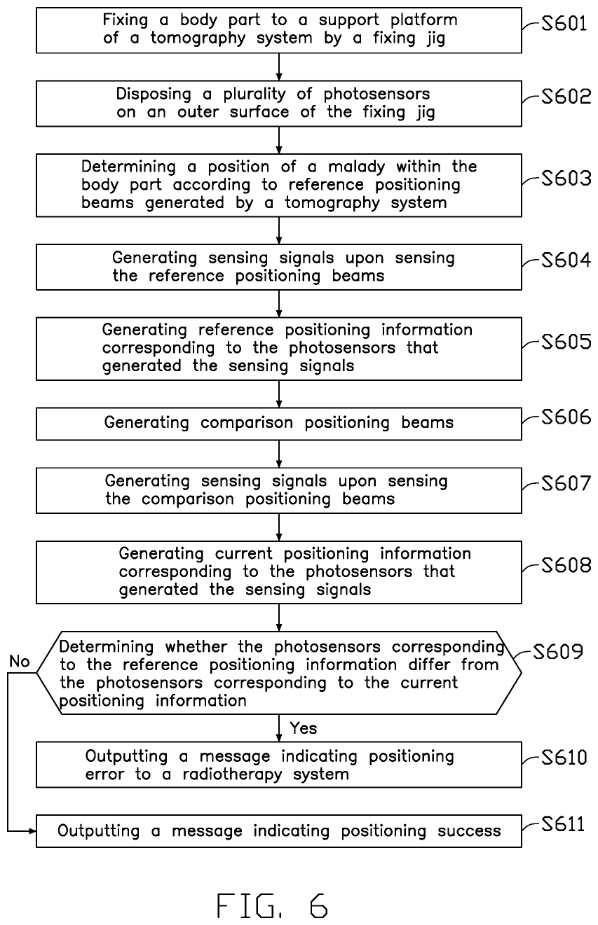

FIG. 6 shows a flowchart of an embodiment of a positioning method. The method is provided by way of embodiment, as there are a variety of ways to carry out the method. The method described below can be carried out using the configurations illustrated in FIGS. 1-4B, for example, and various elements of these figures are referenced in explaining the example method. Each block shown in FIG. 6 represents one or more processes, methods, or subroutines carried out in the example method. Furthermore, the illustrated order of blocks is by example only, and the order of the blocks can be changed. Additional blocks can be added or fewer blocks can be utilized, without departing from this disclosure.

At block S601, a body part is fixed to a support platform of a tomography system by a fixing jig (such as the fixing jig 131).

At block S602, a plurality of photosensors (such as the photosensors 132) are disposed on an outer surface of the fixing jig.

At block S603, a position of a malady (such as a tumor) within the body part is determined according to reference positioning beams generated by a tomography system (such as the tomography system 11).

At block S604, the photosensors generate sensing signals upon sensing the reference positioning beams.

At block S605, a processing unit (such as the processing unit 133), upon receiving the sensing signals from the photosensors, generate reference positioning information corresponding to the photosensors that generated the sensing signals. The reference positioning information may be coordinate information of the photosensors. At this time, the processing unit operates in a recording mode.

At block S606, a comparison positioning beam system (such as the comparison positioning beam system 122) of a radiotherapy system (such as the radiotherapy system 12) generates comparison positioning beams. In one embodiment, the body part can be secured to a treatment platform (such as the treatment platform 121) of the radiotherapy system by the same fixing jig.

At block S607, the photosensors generate sensing signals upon sensing the comparison positioning beams.

At block S608, the processing unit, upon receiving the sensing signals from the photosensors, generates current positioning information corresponding to the photosensors that generated the sensing signals. At this time, the processing unit operates in a comparison mode.

At block S609, the processing unit determines whether the photosensors corresponding to the reference positioning information differs from the photosensors corresponding to the current positioning information. In some embodiments, the reference positioning information and the current positioning information include identification information of the corresponding photosensors. When the processing unit determines that the photosensors corresponding to the reference positioning information differs from the photosensors corresponding to the current positioning information, block S610 is implemented. When the processing unit determines that the photosensors corresponding to the reference positioning information are the same as the photosensors corresponding to the current positioning information, block S611 is implemented.

At block S610, the processing unit outputs a message indicating positioning error to the radiotherapy system. The positioning result may include the reference positioning information and the current positioning information. Upon receiving the positioning result from the processing unit, the radiotherapy system moves the treatment platform based on the positioning result.

At block S611, the processing unit outputs a message indicating positioning success to a user input and output interface (such as the user input and output interface 135) for display to inform an operator, such as a radiotherapist.

In summary, the reference positioning information generated by the processing units can be compared to position the affected body part, so that artificial misjudgment can be avoided. In addition, by determining whether the photosensors corresponding to the reference positioning information differ from the photosensors corresponding to the current positioning information, a positioning accuracy is further ensured.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A positioning device for positioning a body part of a patient for radiotherapy, the positioning system comprising:
a fixing jig configured to cover and conform to a shape of the body part;
at least one photosensor disposed on an outer surface of the fixing jig and configured to generate a sensing signal upon sensing a positioning beam; and
a processing unit operable in a recording mode and a comparison mode, the recording mode to receive the sensing signal of the at least one photosensor and generate reference positioning information according to the sensing signal, and the comparison mode to receive the sensing signal of the at least one photosensor and generate comparison positioning information according to the sensing signal; wherein:
the processing unit is configured to compare the reference positioning information to the comparison positioning information and output a positioning result according to a result of comparison.

2. The positioning device of claim 1 further comprising a signal converter configured to convert the sensing signals generated by the photosensors into digital signals.

3. The positioning device of claim 1 further comprising a user input and output interface, wherein:
the processing unit controls the user input and output interface to display the positioning result.

4. The positioning device of claim 1 comprising a plurality of photosensors, wherein:
each of the plurality of photosensors generates a corresponding sensing signal upon sensing the positioning beam;
the processing unit compares the reference positioning information of each of the plurality of photosensors to the comparison positioning information of the plurality of photosensors.

5. A positioning system for positioning a body part of a patient for radiotherapy, the positioning system comprising:
a positioning device comprising:
at least one photosensor disposed on an outer surface of the body part and configured to generate a sensing signal upon sensing a positioning beam; and
a processing unit operable in a recording mode and a comparison mode, the recording mode to receive the sensing signal of the at least one photosensor and generate reference positioning information according to the sensing signal, and the comparison mode to receive the sensing signal of the at least one photosensor and generate comparison positioning information according to the sensing signal, the processor configured to compare the reference positioning information to the comparison positioning information and output a positioning result according to a result of comparison; and
a radiotherapy system comprising a treatment platform; wherein:
the radiotherapy system is configured to move or not move the treatment platform according to the positioning result.

6. The positioning system of claim 5 further comprising a tomography system, wherein:
the tomography system comprises a reference positioning beam system;
the radiotherapy system further comprises a comparison positioning beam system;
the tomography system emits reference positioning beams to the at least one photosensor, and the processing unit generates the reference positioning information according to the sensing signal generated by the at least one photosensor in response to sensing the reference positioning beams;
the radiotherapy system emits comparison positioning beams to the at least one photosensor, and the processing unit generates the comparison positioning information according to the sensing signal generated by the at least one photosensor in response to sensing the comparison positioning beams.

7. The positioning system of claim 6, wherein:
the positioning device comprises a plurality of photosensors, wherein:
each of the plurality of photosensors generates a corresponding sensing signal upon sensing the reference positioning beam and the comparison positioning beam;
the processing unit compares the reference positioning information of each of the plurality of photosensors to the comparison positioning information of the plurality of photosensors.

8. A positioning method for positioning a body part for radiation treatment, the method comprising:
fixing the body part to a support platform of a tomography system by a fixing jig;
disposing a plurality of photosensors on an outer surface of the fixing jig;
emitting, by the tomography system, reference positioning beams to the plurality of photosensors;
generating, by the plurality of photosensors, sensing signals upon sensing the reference positioning beams;
generating, by a processing unit upon receiving the sensing signals from the photosensors, reference positioning information corresponding to the photosensors that generated the sensing signals;
fixing the body part to a treatment platform of a radiation treatment system;
emitting, by the radiation treatment system, comparison positioning beams to the plurality of photosensors;
generating, by the plurality of photosensors, sensing signals upon sensing the comparison positioning beams;
generating, by the processing unit upon receiving the sensing signals from the photosensors, comparison positioning information corresponding to the photosensors that generated the sensing signals; and
determining whether the reference positioning information differs from the comparison positioning information and outputting a positioning result.

9. The positioning method of claim 8, wherein:
a position of a malady within the body part is determined according to the reference positioning beams.

10. The positioning method of claim 8 further comprising:
moving the treatment platform according to the comparison information when the reference positioning information differs from the comparison positioning information

11. The positioning method of claim 8, wherein:
the positioning result is output to a user input and output interface.
